(19) United States
(12) Patent Application Publication
(10) Pub. No.: US 2025/0099772 A1
(43) Pub. Date: Mar. 27, 2025

(54) CARDIAC PACING ARRANGEMENT

(71) Applicant: BIOTRONIK SE & CO. KG, Berlin (DE)

(72) Inventors: Andreas KUTSAM, Berlin (DE); Klaus SCHMOLINSKY, Berlin (DE)

(73) Assignee: BIOTRONIK SE & CO. KG, Berlin (DE)

(21) Appl. No.: 18/833,172

(22) PCT Filed: Jan. 19, 2023
(86) PCT No.: PCT/EP2023/051234

§ 371 Date: Jul. 25, 2024

(30) Foreign Application Priority Data
Feb. 7, 2022 (EP) 22155404.1

(51) Int. Cl. A61N 1/375; A61N 1/365; A61N 1/37; A61N 1/372

(52) U.S. Cl. CPC A61N 1/3756; A61N 1/365; A61N 1/3702; A61N 1/371; A61N 1/37211; A61N 1/37512

(57) ABSTRACT

Cardiac pacing arrangement comprising first and second leadless pacing devices, wherein the second leadless pacing device is operated in standby or operational states. During operation, the cardiac pacing arrangement carries out the following steps: a) determining whether a condition for transferring the second leadless pacing device from the standby to the operational state is fulfilled, wherein the condition is chosen from the group consisting of i) delivering N consecutive stimulating pulses (N is an integer from 2-10), ii) non-detection of any stimulation pulse over a predeterminable time period (0.5-72 hours) and detection of an existing intrinsic rhythm of the patient at the same time, and iii) detecting M consecutive non-capturing stimulation pulses (M is an integer from 2-10); and b) transferring the second leadless pacing device from the standby to the operational state if the condition is fulfilled.

### CROSS-REFERENCE TO RELATED APPLICATIONS

This application is the United States National Phase under 35 U.S.C. § 371 of PCT International Patent Application No. PCT/EP2023/051234, filed on Jan. 19, 2023, which claims the benefit of European Patent Application No. 22155404.1, filed on Feb. 7, 2022, the disclosures of which are hereby incorporated by reference herein in their entireties.

### TECHNICAL FIELD

The present invention relates to a cardiac pacing arrangement according to the preamble of claim 1, to a method for controlling such a cardiac pacing arrangement according to the preamble of claim 9, and to a computer program product according to the preamble of claim 10.

### BACKGROUND

In recent years, leadless pacemakers have received increasing attention. Leadless pacemakers, in contrast to pacemakers implanted subcutaneously using leads extending transvenously into the heart, avoid leads in that the pacemaker device itself is implanted into the heart. Leadless pacemakers typically have the shape of a capsule for implantation into cardiac tissue, e.g., cardiac tissue of the right atrium and/or right ventricle. Such leadless pacemakers exhibit the inherent advantage of not using leads, which can reduce risks for the patient involved with leads transvenously accessing the heart, such as the risk of pneumothorax, infection of the pacing system, lead failure (fracture or insulation defect) and the like.

Leadless pacemakers may specifically be designed for implantation in the right ventricle and/or right atrium and, in this case, during implantation are preferably placed in or on the septal or apical region of the right ventricle. Leadless Pacemakers are not conducive to using radio frequency (RF) telemetry to do remote monitoring due to extreme low power design, but an inductive communication interface can be exploited to achieve a patient triggered remote interrogation when the circumstances warrant it.

If a leadless pacemaker has been implanted for longer period of time, it can regularly not be explanted anymore. Rather, when reaching the end of its lifetime (also referred to as “end of lifetime”, EOL or “end of service”, EOS), the leadless pacemaker will remain within the heart of the patient. Further cardiac stimulation will be performed by another pacemaker (leadless or lead-based) to be implanted into the same heart.

Over an extended period of time, a plurality of leadless pacemakers may be implanted within the patient's heart. The procedure of changing the stimulation device/unit from an old leadless pacemaker to a new leadless pacemaker is also referred to as box change procedure. A temporal security buffer for such box change is necessary to avoid any periods in which no pacing would be possible. Therefore, any subsequent (new) leadless pacemaker needs to be implanted prior to the end of service of the preceding (old) leadless pacemaker. Thus, slightly before or after reaching an elective replacement indicator (ERI) of a preceding leadless pacemaker, the respective succeeding leadless pacemaker will be implanted. Consequently, implantation of such succeeding leadless pacemaker is carried out when the battery of the preceding leadless pacemaker is not yet fully discharged. As a result, a part of the battery capacity of the preceding (old) leadless pacemaker is wasted upon deactivation of the preceding leadless pacemaker and activation of the succeeding (new) leadless pacemaker.

International Publication No. WO 2015/095818 A1 describes an approach in which a box change procedure is performed with an improved power usage. For this purpose, a novel leadless pacemaker is implanted and initially kept in a dormant state while the old leadless pacemaker is still active. At a later stage, the old leadless pacemaker sends a signal to the novel leadless pacemaker to activate the novel leadless pacemaker. In doing so, the cumulative lifetime of the old and the new leadless pacemaker is higher than in case of applying standard box change procedures. However, the procedure described in International Publication No. WO 2015/095818 A1 requires a compatibility of the old leadless pacemaker and the new leadless pacemaker to enable an activation of the new leadless pacemaker.

The present disclosure is directed toward overcoming one or more of the above-mentioned problems, though not necessarily limited to embodiments that do.

### SUMMARY

It is an object of the present invention to provide a system and a method for a box change procedure of a leadless pacemaker that uses the electrical power of an old leadless pacemaker and a new leadless pacemaker to full capacity, maintains high patient safety and can also be applied in case of non-compatible leadless pacemakers.

At least this object is achieved with a cardiac pacing arrangement having the claim elements of claim 1. Such a cardiac pacing arrangement comprises a first leadless pacing device and a second leadless pacing device.

The first leadless pacing device comprises a first processor, a first memory unit, a first stimulation unit and a first detection unit. The first stimulation unit is configured to stimulate a human or animal heart. The first detection unit is configured to detect an electric signal of the same heart.

The second leadless pacing device comprises a second processor, a second memory unit, a second stimulation unit, and a second detection unit. The second stimulation unit is configured to stimulate the same heart as the first stimulation unit. Likewise, the second detection unit is configured to detect an electric signal of the same heart as the first detection unit. Thus, both the first leadless pacing device and the second leadless pacing device serve for sensing and pacing one and the same heart of a human or animal patient.

The second leadless pacing device is furthermore configured to be operated in one of at least two functional states. The first functional state is a standby state (also referred to as dormant state). The second functional state is an operational state (also referred to as permanent state).

Other pacing devices may also be used instead of the leadless pacing devices, e.g., lead-based pacing devices or pacemakers implanted subcutaneously using leads extending transvenously into the heart.

According to an aspect of the present invention, the second memory unit comprises a second computer-readable program that causes the second processor to perform the steps explained in the following when executed on the second processor.

First, it is determined whether a condition for transferring the second leadless pacing device from the standby state to the operational state is fulfilled. This condition is chosen from one of the conditions explained in the following.

A first condition is fulfilled if N consecutive stimulating pulses are delivered by the second stimulation unit. In this context, N is an integer lying in a range of from 2 to 50, in particular of from 2 to 45, in particular of from 2 to 40, in particular of from 2 to 35, in particular of from 2 to 30, in particular of from 2 to 25, in particular of from 2 to 20, in particular of from 2 to 15, in particular of from 2 to 10, in particular of from 2 to 8, in particular of from 2 to 5. If the second stimulating unit needs to deliver this amount of consecutive stimulating pulses, this is a direct pointer that the first leadless pacing device (i.e., the old leadless pacing device that is to be replaced by the second (new) leadless pacing device) does no longer stimulate the patient's heart due to a discharge of its battery. At the same time, the patient requires the stimulation for which reason the second leadless pacing device has already delivered a plurality of consecutive stimulation pulses in its standby state.

A second condition is fulfilled if the second detection unit was not able to detect any stimulation pulse delivered by the first stimulation unit over a predetermined time period lying in a range of from 0.5 hours to 72 hours, in particular of from 1 hours to 60 hours, in particular of from 1 hours to 48 hours. If not a single stimulation pulse of the first leadless pacing device has been detected over such a long time period, this is a clear pointer that the battery of the first leadless pacing device is discharged, as a result of which the first leadless pacing device is no longer able to stimulate the patient's heart and also cannot perform any automatic tests (such as capture threshold tests). At the same time, the patient has a sufficiently vital intrinsic cardiac rhythm so that a backup stimulation by the second leadless pacing device is currently not necessary.

A third condition is fulfilled if M consecutive non-capturing stimulation pulses delivered by the first stimulation unit are detected with the second detection unit. In this context, M is an integer lying in a range of from 2 to 50, in particular of from 2 to 40, in particular of from 2 to 30, in particular of from 2 to 20, in particular of from 2 to 10, in particular of from 2 to 8, in particular of from 2 to 5. Detection of such non-capturing stimulation pulses is a clear pointer that the battery capacity of the first leadless pacing device is too low to deliver stimulation pulses exceeding the stimulation threshold. However, stimulation pulses lying below the stimulation threshold will result in non-capturing events (i.e., no physiologic responses to the applied stimulation pulses). Since non-capturing stimulation pulses result in a non-stimulation of the patient's heart, the further cardiac stimulation needs to be performed by the second leadless pacing device.

In a second step performed by the second processor, the second leadless pacing device is transferred from the standby state to the operational state if at least one of the conditions explained above is fulfilled. Then, the second leadless pacing device will not only be able to provide a backup stimulation to the patient, but rather to employ its full functionality within the patient's heart. By transferring the second leadless pacing device into the operational state, the second leadless pacing device will take over all the relevant stimulation activities hitherto performed by the first leadless pacing device. Thus, transferring the second leadless pacing device into the operational state constitutes the last step of the whole box change procedure by which the pacing functionalities are transferred from the first leadless pacing device to the second leadless pacing device.

As long as the first leadless pacing device properly functions, no full operation of the second leadless pacing device is necessary. Rather by keeping the second leadless pacing device in the standby state as long as possible, the battery capacity of the second leadless pacing device is saved and will increase the cumulated lifetime of the first leadless pacing device and the second leadless pacing device. Consequently, the implantation of a third and any subsequent leadless pacing device needs only to be performed at a later time point than in case of deactivating a first leadless pacing device upon implanting a fully operational second leadless pacing device. At the same time, the patient safety is particularly high since the second leadless pacing device is also able to perform a backup stimulation in its backup state if the first leadless pacing device is no longer able to perform an appropriate cardiac stimulation.

While it is possible that the first leadless pacing device and the second leadless pacing device are compatible devices that are able to communicate with each other, this is not a prerequisite. Rather, in an embodiment, the first leadless pacing device and the second leadless pacing device are non-compatible devices. The presently claimed cardiac pacing arrangement is fully functional even in this embodiment since the second leadless pacing device is not dependent on a specific dataset or information provided by the first leadless pacing device to be transferred from its standby state to its operational state. Rather, the second leadless pacing device detects a malfunction of the first leadless pacing device (in particular caused by an exhausted or discharged battery of the first leadless pacing device) and will then be automatically activated. Since a compatibility between the first leadless pacing device and the second leadless pacing device is not necessary, the presently claimed cardiac pacing arrangement can be universally applied regardless on the type and manufacturer of the first leadless pacing device.

In an embodiment, the second leadless pacing device is configured such that an operation of the second leadless pacing device in the standby state enables a backup stimulation of the heart with a backup stimulation rate being lower than a regular or lowest stimulation rate applied by the first stimulating unit of the first leadless pacing device, even or optional during tests or application of hysteresis. By such a configuration, any conflict between the first leadless pacing device and the second leadless pacing device is prevented that could otherwise occur while the first leadless pacing device is still active. To give an example, the backup stimulation may be employed in a VVI mode (ventricular pacing, ventricular sensing and inhibition of pacing in case of an intrinsic cardiac activity has been detected) at a rate lying in a range of from 30 bpm to 60 bpm, in particular of from 30 ppm to 55 bpm, in particular from of from 35 ppm to 50 ppm. In contrast, the regular or lowest stimulation rate applied by the first stimulating unit of the first cardiac leadless pacing device, even or optional during tests or application of hysteresis, is higher than the applied backup stimulation rate. Consequently, as long as the first leadless pacing device is active, its stimulation will dominate the cardiac rhythm of the patient, whereas the second leadless pacing device will not be put into action since the stimulation rate applied by the first leadless pacing device is higher than its own backup stimulation rate.

In an embodiment, the second leadless pacing device is designed such that at least some, in particular all, electricity-consuming elements or operations of the second leadless pacing device are deactivated if the second leadless pacing device is operated in the standby state. In this context, only those electricity-consuming elements or operations of the second leadless pacing device are deactivated that are not necessary for delivering stimulation pulses by the second stimulation unit and/or monitoring the cardiac rate to detect the necessity for the stimulation/to monitor triggers. The energy consumption of the second leadless pacing device is reduced or significantly reduced by such deactivation of selected or all not fully necessary electricity-consuming elements or operations. This extends its lifetime and thus the lifetime of the whole cardiac pacing arrangement.

In an embodiment, the electricity-consuming elements or operations of the second leadless pacing device that are not necessary for delivering stimulation pulses by the second stimulation unit are one or more of sensors being different/other than the second detection unit (which is necessary for determining whether a stimulation with the second stimulation unit is to be performed), statistic calculations relating to sensed electric signals and/or delivered pacing pulses, and measurements, e.g., automatic capture threshold test measurements or impedance measurements.

Additionally or alternatively, the electricity-consuming elements or operations of the second leadless pacing device that are not necessary for delivering stimulation pulses by the second stimulation unit are chosen from the group consisting of sensors like statistic calculations relating to sensed electric signals, automatic capture threshold test measurements, impedance and other measurements—in general: other than the second detection unit and/or pacing pulses delivery unit and/or stimulation unit.

While all of these elements or operations will be needed in an operational state to ensure proper functioning of the second leadless pacing device as well as proper documentation of its functioning, these element and operations will not be needed in the standby mode of the second leadless pacing device. This is due to the fact that comparable elements and operations will be activated or performed by the first leadless pacing device still being active during that standby phase of the second leadless pacing device.

In an embodiment, the second leadless pacing device is immediately transferred from the standby state to the operational state, i.e., on a beat-to-beat basis. Such immediate transfer means that all functions and functionalities of the operational state are activated from one heartbeat to another heartbeat. In another embodiment, the transfer of the second leadless pacing device from the standby state of the operational state is performed in a stepwise manner, i.e., not directly from one heartbeat to the next heartbeat, but rather over a period of a plurality of heartbeats. Such stepwise transfer from the standby state to the operational state may be more physiologic than a beat-to-beat transfer.

In an embodiment, the second computer-readable program causes the second processor to send information indicating that the second leadless pacing device is operated in the operational state to the first leadless pacing device upon being transferred from the standby state to the operational state. Such information can be provided from the second leadless pacing device to the first leadless pacing device by an inter-body communication or via a specific, unique, predefined stimulation sequence. Such back-channel communication between the second leadless pacing device and the first leadless pacing device requires a compatibility of the first leadless pacing device and the second leadless pacing device to enable the first leadless pacing device understanding the information (or commands) provided by the second leadless pacing device. Thus, this embodiment is typically combined with an embodiment in which the first leadless pacing device and the second leadless pacing device are compatible with respect to data/information exchange.

In an embodiment, the first leadless pacing device is compatible to the second leadless pacing device, i.e., the first leadless pacing device understands information sent by the second leadless pacing device, wherein the second leadless pacing device does not necessarily understand information sent by the first leadless pacing device. Expressed in other words, this embodiment requires a unidirectional compatibility, but not necessarily a bidirectional compatibility between the first leadless pacing device and the second leadless pacing device.

In an embodiment, the first memory unit comprises a first computer-readable program that causes the first processor, when being executed on the first processor, to transfer the first leadless pacing device from an operational state to a standby state upon receiving the information indicating that the second leadless pacing device is operated in the operational state. Thus, the back channel between the second leadless pacing device and the first leadless pacing device can be used to actively change the functional state of the first leadless pacing device. Such an active change of the functional state may be helpful even in case that the battery capacity of the first leadless pacing device is too low to properly stimulate the patient's heart. This is because the first leadless pacing device can possibly perform specific tasks in its standby state that may help saving energy of the second leadless pacing device. Then, the overall lifetime of the cardiac pacing arrangement is further increased and the time point of implanting a third or further leadless pacing device is further shifted to the future.

In an embodiment, the first leadless pacing device is designed such that an operation of the first leadless pacing device in the standby state enables the first leadless pacing device to perform statistic calculations relating to sensed electric signals. Then, the computing or power capacity necessary for performing such statistic calculations does not need to be invested by the second leadless pacing device. Rather, the battery of the first leadless pacing device (even being too exhausted to properly stimulate the patient's heart) can be further exploited.

In an embodiment, the first memory unit comprises a first computer-readable program that causes the first processor, when being executed on the first processor, to transfer the first leadless pacing device from an operational state to a deactivated state upon receiving the information indicating that the second leadless pacing device is operated in the operational state. In contrast to the precedingly explained embodiment, this embodiment does not transfer the first leadless pacing device into a standby state, but rather into a deactivated (off) state in which it cannot perform any further tasks. This helps in enhancing the safety of the cardiac pacing arrangement since it prevents the risk that the first leadless pacing device is—due to low battery capacity—not in a position to perform any further tasks (like provided in the standby state of the first leadless pacing device). In any case, the first leadless pacing device will be transferred into the deactivated state only if its battery capacity is too low to further stimulate the patient's heart. In such a case, the battery of the first leadless pacing device is deemed to be fully used.

In an embodiment, the cardiac pacing arrangement is a pacing arrangement comprising more than two leadless pacing devices. To give an example, it may comprise a first atrial leadless pacing device, a second atrial leadless pacing device, a first ventricular leadless pacing device, and a second ventricular leadless pacing device. In such a case, the second atrial leadless pacing device will only be transferred from its standby state to its operational state in case that one of the above-mentioned conditions (first, second and/or third) is fulfilled with respect to the first atrial leadless pacing device. Likewise, the second ventricular leadless pacing device will only be transferred from its standby state to its operational state in case that one of the above-mentioned conditions are fulfilled with respect to the first ventricular leadless pacing device.

In case of more than two leadless pacing devices in the cardiac pacing arrangement, it is also possible that individual leadless pacing devices are to be replaced by novel leadless pacing devices at different time points. Therefore, such cardiac pacing arrangement may comprise a different number of leadless pacing devices over time.

In an embodiment, the two functional states (i.e., the standby state and the operational state) are represented by programs of the second leadless pacing device that are transferred by a programmer to the second leadless pacing device in form of an inseparable composite command. If this composite command is correctly received by the second leadless pacing device, it will provide a confirmation on this receipt. If the composite command has not been correctly received by the second leadless pacing device, it will be necessary to transfer the composite command once again or with appropriate repetitions. By applying such programming of the second leadless pacing device, it is guaranteed that the standby state will not be the only functional state that can be applied by the second leadless pacing device, but that the second leadless pacing device will necessarily also be able to be operated in the operational state. This further enhances the reliability and safety of the whole cardiac pacing system.

In an aspect, the present invention relates to a method for controlling a cardiac pacing arrangement according to the preceding explanations. This method comprises the steps explained in the following.

First, it is determined whether a condition for transferring the second leadless pacing device from the standby state to the operational state is fulfilled. This condition is chosen from one of the conditions explained in the following.

A first condition is fulfilled if N consecutive stimulating pulses are delivered by the second stimulation unit. In this context, N is an integer lying in a range of from 2 to 50, in particular of from 2 to 45, in particular of from 2 to 40, in particular of from 2 to 35, in particular of from 2 to 30, in particular of from 2 to 25, in particular of from 2 to 20, in particular of from 2 to 15, in particular of from 2 to 10, in particular of from 2 to 8, in particular of from 2 to 5.

A second condition is fulfilled if the second detection unit was not able to detect any stimulation pulse delivered by the first stimulation unit over a predetermined time period lying in a range of from 0.5 hours to 72 hours, in particular of from 1 hour to 60 hours, in particular of from 1 hour to 48 hours and optional detection of an existing intrinsic rhythm of the patient at the same time.

A third condition is fulfilled if M consecutive non-capturing stimulation pulses delivered by the first stimulation unit are detected with the second detection unit. In this context, M is an integer lying in a range of from 2 to 50, in particular of from 2 to 45, in particular of from 2 to 40, in particular of from 2 to 35, in particular of from 2 to 30, in particular of from 2 to 20, in particular of from 2 to 15, in particular of from 2 to 10, in particular of from 2 to 8, in particular of from 2 to 5.

Secondly, the second leadless pacing device is transferred from the standby state to the operational state if at least one of the conditions explained above is fulfilled.

In an aspect, the present invention relates to computer program product comprising computer-readable code that causes a processor to perform the steps explained in the following when executed on the processor.

First, it is determined whether a condition for transferring the second leadless pacing device from the standby state to the operational state is fulfilled. This condition is chosen from one of the conditions explained in the following.

A first condition is fulfilled if N consecutive stimulating pulses are delivered by a second stimulation unit of the second leadless pacing device. In this context, N is an integer lying in a range of from 2 to 50, in particular of from 2 to 45, in particular of from 2 to 40, in particular of from 2 to 35, in particular of from 2 to 30, in particular of from 2 to 25, in particular of from 2 to 20, in particular of from 2 to 15, in particular of from 2 to 10, in particular of from 2 to 8, in particular of from 2 to 5.

A second condition is fulfilled if a second detection unit of the second leadless pacing device was not able to detect any stimulation pulse delivered by the first stimulation unit over a predetermined time period lying in a range of from 0.5 hours to 72 hours, in particular of from 1 hour to 60 hours, in particular of from 1 hour to 48 hours, but was optionally able to detect an existing intrinsic rhythm of the patient at the same time.

A third condition is fulfilled if M consecutive non-capturing stimulation pulses delivered by a first stimulation unit of a first leadless pacing device are detected with the second detection unit. In this context, M is an integer lying in a range of from 2 to 50, in particular of from 2 to 45, in particular of from 2 to 40, in particular of from 2 to 35, in particular of from 2 to 30, in particular of from 2 to 20, in particular of from 2 to 15, in particular of from 2 to 10, in particular of from 2 to 8, in particular of from 2 to 5.

Secondly, the second leadless pacing device is transferred from the standby state to the operational state if at least one of the conditions explained above is fulfilled.

In an aspect, the present invention relates to medical method for stimulating a patient's heart with cardiac pacing arrangement according to the preceding explanations. This method comprises the steps explained in the following.

First, it is determined whether a condition for transferring the second leadless pacing device from the standby state to the operational state is fulfilled. This condition is chosen from one of the conditions explained in the following.

A first condition is fulfilled if N consecutive stimulating pulses are delivered by the second stimulation unit. In this context, N is an integer lying in a range of from 2 to 50, in particular of from 2 to 45, in particular of from 2 to 40, in particular of from 2 to 35, in particular of from 2 to 30, in particular of from 2 to 25, in particular of from 2 to 20, in particular of from 2 to 15, in particular of from 2 to 10, in particular of from 2 to 8, in particular of from 2 to 5.

A second condition is fulfilled if the second detection unit was not able to detect any stimulation pulse delivered by the first stimulation unit over a predetermined time period lying in a range of from 0.5 hours to 72 hours, in particular of from 1 hour to 60 hours, in particular of from 1 hour to 48 hours, but was optionally able to detect an existing intrinsic rhythm of the patient at the same time.

A third condition is fulfilled if M consecutive non-capturing stimulation pulses delivered by the first stimulation unit are detected with the second detection unit. In this context, M is an integer lying in a range of from 2 to 50, in particular of from 2 to 45, in particular of from 2 to 40, in particular of from 2 to 35, in particular of from 2 to 30, in particular of from 2 to 20, in particular of from 2 to 15, in particular of from 2 to 10, in particular of from 2 to 8, in particular of from 2 to 5.

Secondly, the second leadless pacing device is transferred from the standby state to the operational state if at least one of the conditions explained above is fulfilled.

Finally, at least one stimulation pulse is delivered to the patient's heart by the second stimulation unit and/or another stimulation unit or pacing device.

In an embodiment, at least one of the stimulation units and pacing devices mentioned may be a multi-chamber unit/device.

Further, additional (external) triggers for stimulation by a pacing device and and/or stimulation unit are possible, but such additional triggers only functioning in addition to the triggers mentioned above (triggers may be all devices/units triggering a stimulation). Two examples of such additional triggers are: a) triggering after expiry of a pre-programmed timer in the new (preferably first) leadless pacing device, in order to guarantee the latest possible and/or fixed time for the activation of the new leadless pacing device: and b) an external command to a new leadless pacing device, e.g., by applying/placing a magnet close to the pacing device or sending a command using an external device (remote assistant or any external device capable of doing so).

All embodiments of the cardiac pacing arrangement can be combined in any desired manner and can be transferred either individually or in any arbitrary combination to the described methods and the described computer program product. Likewise, all embodiments of the described methods can be combined in any desired manner and can be transferred either individually or in any arbitrary combination to the cardiac pacing arrangement, to the respective other method, and to the computer program product. Finally, all embodiments described with respect to the computer program product can be combined in any desired manner and can be transferred either individually or in any arbitrary combination to the cardiac pacing arrangement or to any of the described methods.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

Further details of aspects of the present invention will be explained in the following making reference to an exemplary embodiment and a FIGURE. In the FIGURE:

FIG. 1 shows a schematic timeline of the lifetime of two leadless pacing devices of a cardiac pacing arrangement.

### DETAILED DESCRIPTION

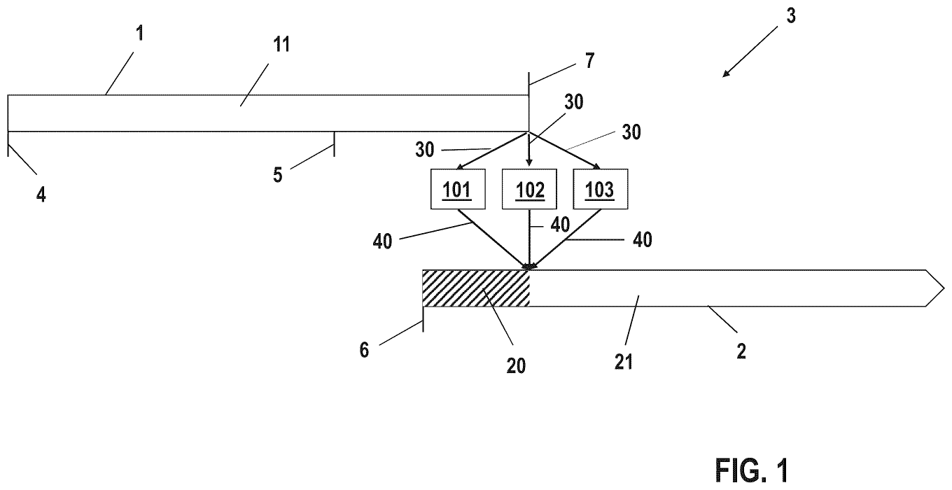

FIG. 1 schematically shows a timeline representing the lifetime of a first leadless pacemaker 1 serving as first leadless pacing device and a second leadless pacemaker 2 serving as second leadless pacing device. The first leadless pacemaker 1 and the second leadless pacemaker 2 form a cardiac pacing arrangement 3.

At a first implantation time point 4, the first leadless pacemaker 1 is implanted into the heart of a human or animal patient. Shortly after reaching an elective replacement indicator (ERI) 5, the second leadless pacemaker 2 is implanted into the heart of the same patient at a second implantation time point 6. Measurements and tests necessary for proper operation of the second leadless pacemaker 2 are performed upon or directly after its implantation. The second leadless pacemaker 2 is provided with two functional modes, namely, a waiting program 20 (or dormant state or standby state) serving as standby state and a permanent program 21 serving as operational state.

The first leadless pacemaker 1 is normally active in its permanent program 11, while the second leadless pacemaker 2 is already implanted and employs its waiting program 20. This waiting program 20 enables backup pacing for safety reasons, wherein all other potentially electricity-consuming tasks and elements are deactivated. To give an example, the second leadless pacemaker 2 does not calculate any statistics in its waiting program 20.

At a generally unknown time point 7, the first leadless pacemaker 1 stops its pacing without external trigger to do so due to an exhaustion of its battery. The second leadless pacemaker 2 recognizes this stop of pacing by determining 30 whether at least one of a first condition 101, a second condition 102, and a third condition 103 is fulfilled. If this is the case, the second leadless pacemaker 2 automatically carries out a step of transferring 40 its functional state from the waiting program 20 to the permanent program 21. While applying this permanent program 21, the second leadless pacemaker is able to perform any stimulation and auxiliary tasks that are regularly performed during standard stimulation. In doing so, the leadless pacemaker 2 takes over the full stimulation functionality from the first leadless pacemaker 1 at the generally unknown time point 7 if the first condition 101, the second condition 102 and/or the third condition 103 is fulfilled.

The first condition 101 is fulfilled if N consecutive stimulating pulses are delivered by the second stimulating unit (with N being 2 to 50 or N being 2 to 10). The second condition 102 is fulfilled if the second detection unit is not able to detect any stimulation pulse delivered by the first stimulation unit over a predetermined period of time that has been set to 24 hours in the embodiment depicted in FIG. 1. The third condition 103 is fulfilled if the second detection unit detects M consecutive non-capturing stimulation pulses delivered by the first stimulation unit, wherein M is between 2 and 50 or M is between 2 and 10.

These very specific conditions 101, 102, 103 ensure that the second leadless pacemaker 2 automatically changes its operation by switching from the waiting program 20 to the permanent program 21 at the generally unknown time point 7 at which the first leadless pacemaker 1 is no longer able to perform proper stimulation of the heart into which it is implanted. No external trigger is necessary to be received by the second leadless pacemaker 2 for this automatic change. Thus, the setup of the cardiac pacing arrangement 3 ensures the maximum possible lifetime of the first leadless pacemaker 1 and the second leadless pacemaker 2 so that an implantation of a third leadless pacemaker can be significantly postponed. The setup does not require a compatibility between the first leadless pacemaker 1 and the second leadless pacemaker 2. Furthermore, the setup prevents the risk that any command sent from the first leadless pacemaker 1 to the second leadless pacemaker 2 is not received by the second leadless pacemaker 2 or is incorrectly interpreted by the second leadless pacemaker 2. Rather, a full activation of the second leadless pacemaker 2 will automatically be triggered by the end of service of the first leadless pacemaker 1 at the generally unknown time point 7.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. Cardiac pacing arrangement comprising a first leadless pacing device and a second leadless pacing device,
wherein the first leadless pacing device comprises a first processor, a first memory unit, a first stimulation unit configured to stimulate a human or animal heart, and a first detection unit configured to detect an electric signal of the same heart, wherein the second leadless pacing device comprises a second processor, a second memory unit, a second stimulation unit configured to stimulate the same heart as the first stimulation unit, and a second detection unit configured to detect an electric signal of the same heart as the first detection unit,
wherein the second leadless pacing device is configured to be operated in a standby state or in an operational state,
wherein
in that the second memory unit comprises a second computer-readable program that causes the second processor to perform the following steps when executed on the second processor:
a) determining whether a condition for transferring the second leadless pacing device from the standby state to the operational state is fulfilled, wherein the condition is chosen from the group consisting of i) delivering N consecutive stimulating pulses by the second stimulating unit, wherein N is an integer lying in a range of from 2 to 10, ii) non-detection, with the second detection unit, of any stimulation pulse delivered by the first stimulation unit over a predeterminable time period lying in a range of from 0.5 hours to 72 hours and detection of an existing intrinsic rhythm of the patient at the same time, and iii) detecting, with the second detection unit, M consecutive non-capturing stimulation pulses delivered by the first stimulation unit, wherein M is an integer lying in a range of from 2 to 10; and
b) transferring the second leadless pacing device from the standby state to the operational state if the condition is fulfilled.

2. Cardiac pacing arrangement according to claim 1, wherein in that the second leadless pacing device is configured such that an operation of the second leadless pacing device in the standby state enables a backup stimulation of the heart with a backup stimulation rate being lower than a lowest stimulation rate applied by the first stimulating unit of the first leadless pacing device, even during tests or application of hysteresis.

3. Cardiac pacing arrangement according to claim 1, wherein in that the second leadless pacing device is configured such that at least some electricity-consuming elements or operations of the second leadless pacing device that are not necessary for delivering stimulation pulses by the second stimulation unit are deactivated if the second leadless pacing device is operated in the standby state.

4. Cardiac pacing arrangement according to claim 3, wherein in that the electricity-consuming elements or operations of the second leadless pacing device that are not necessary for delivering stimulation pulses by the second stimulation unit are chosen from the group consisting of one or more sensors other than the second detection unit, statistic calculations relating to sensed electric signals and/or delivered pacing pulses, and measurements.

5. Cardiac pacing arrangement according to claim 1, wherein in that the second computer-readable program causes the second processor to send information indicating that the second leadless pacing device is operated in the operational state to the first leadless pacing device upon transferring the second leadless pacing device from the standby state to the operational state.

6. Cardiac pacing arrangement according to claim 5, wherein in that the first memory unit comprises a first computer-readable program that causes the first processor, when executed on the first processor, to transfer the first leadless pacing device from an operational state to a standby state upon receiving the information indicating that the second leadless pacing device is operated in the operational state.

7. Cardiac pacing arrangement according to claim 6, wherein in that the first leadless pacing device is configured such that an operation of the first leadless pacing device in the standby state enables the first leadless pacing device to perform statistic calculations relating to sensed electric signals.

8. Cardiac pacing arrangement according to claim 5, wherein in that the first memory unit comprises a first computer-readable program that causes the first processor, when executed on the first processor, to transfer the first leadless pacing device from an operational state to a deactivated state upon receiving the information indicating that the second leadless pacing device is operated in the operational state.

9. Method for controlling a cardiac pacing arrangement according to claim 1, the method comprising the following steps:
a) determining whether a condition for transferring the second leadless pacing device from the standby state to the operational state is fulfilled, wherein the condition is chosen from the group consisting of i) delivering N consecutive stimulating pulses by the second stimulating unit, wherein N is an integer lying in a range of from 2 to 10, ii) non-detection, with the second detection unit, of any stimulation pulse delivered by the first stimulation unit over a predeterminable time period lying in a range of from 0.5 hours to 72 hours and detection of an existing intrinsic rhythm of the patient at the same time, and iii) detecting, with the second detection unit, M consecutive non-capturing stimulation pulses delivered by the first stimulation unit, wherein M is an integer lying in a range of from 2 to 10; and
b) transferring the second leadless pacing device from the standby state to the operational state if the condition is fulfilled.

10. Computer program product comprising computer-readable code that causes a processor to perform the following steps when executed on the processor:
a) determining whether a condition for transferring a second leadless pacing device from a standby state to an operational state is fulfilled, wherein the condition is chosen from the group consisting of i) delivering N consecutive stimulating pulses by a second stimulating unit of the second leadless pacing device, wherein N is an integer lying in a range of from 2 to 10, ii) non-detection, with a second detection unit of the second leadless pacing device, of any stimulation pulse delivered by a first stimulation unit of a first leadless pacing device over a predeterminable time period lying in a range of from 0.5 hours to 72 hours and detection of an existing intrinsic rhythm of the patient at the same time, and iii) detecting, with the second detection unit, M consecutive non-capturing stimulation pulses delivered by a first stimulation unit of a first leadless pacing device, wherein M is an integer lying in a range of from 2 to 10; and
b) transferring the second leadless pacing device from the standby state to the operational state if the condition is fulfilled.

11. A method for stimulating a patient's heart with a cardiac pacing arrangement according to claim 1, the method comprising the following steps:
a) determining whether a condition for transferring the second leadless pacing device from the standby state to the operational state is fulfilled, wherein the condition is chosen from the group consisting of i) delivering N consecutive stimulating pulses by the second stimulating unit, wherein N is an integer lying in a range of from 2 to 10, ii) non-detection, with the second detection unit, of any stimulation pulse delivered by the first stimulation unit over a predeterminable time period lying in a range of from 0.5 hours to 72 hours and detection of an existing intrinsic rhythm of the patient at the same time, and iii) detecting, with the second detection unit, M consecutive non-capturing stimulation pulses delivered by the first stimulation unit, wherein M is an integer lying in a range of from 2 to 10;
b) transferring the second leadless pacing device from the standby state to the operational state if the condition is fulfilled; and
c) delivering at least one stimulation pulse to the patient's heart by the second stimulation unit.
